(19) United States
(12) Patent Application Publication
(10) Pub. No.: US 2007/0259025 A1
(43) Pub. Date: Nov. 8, 2007

(54) Absorbent antiviral structure

(76) Inventor: Michele C. Strocel, Fenton, MI (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/799,663

(22) Filed: May 2, 2007

Related U.S. Application Data
(60) Provisional application No. 60/746,408, filed on May 4, 2006.
(60) Provisional application No. 60/919,411, filed on Mar. 22, 2007.

(51) Int. Cl. A61K 9/70; A61K 9/50; A61K 31/045

(52) U.S. Cl. 424/443; 424/490; 514/729

(57) ABSTRACT

An absorbent device including an absorbent portion, an impermeable membrane that is coupled to the absorbent portion and an attachment member operably coupled to the absorbent portion. The attachment member is adapted to couple the absorbent device to a human appendage such as a forearm, wrist or upper arm. The absorbent device contains a virucidal agent effective to inactivate viruses that are deposited on or in the absorbent portion.

### RELATED APPLICATIONS

This application claims the benefit of U.S. Provisional Applications No. 60/746,408 filed May 4, 2006 entitled “Cough Cover” and 60/919,411 filed Mar. 22, 2007 entitled “Absorbent Antiviral Structure”, which are incorporated herein in their entirety by reference.

### FIELD OF THE INVENTION

The invention generally relates to absorbent structures. More particularly, the invention relates to antipathogenic or antimicrobial absorbent structures that can be secured to another surface.

### BACKGROUND OF THE INVENTION

The Centers for Disease Control and Prevention recommend that if an individual does not have a tissue to cough or sneeze into, that individual should cough or sneeze into their sleeve. Young children do not understand the concept of blowing their noses and tend to wipe their noses on their sleeves, arms or hands. The accumulation of nasal and oral secretions on the sleeves, arms or hands can be unpleasant, unsightly and provide a source for transmission of infection.

In spite of persistent and commonly held beliefs that colds and other infectious diseases are spread primarily by airborne pathogens or caused by exposure to cold weather or variations in temperature, the most common route of transmission of infectious disease, particularly viral disease is through hand to hand contact. Direct person to person contact is very common among young children who commonly touch one another without much inhibition. Direct person to person contact is less common among adults but still occurs in the form of handshaking, hugging, kissing and so on. Adults more commonly transmit infectious organisms by indirect contact. Indirect transmission commonly occurs via nasal or oral secretions that are gotten on the hands and then transferred to commonly used surfaces, such as doorknobs, elevator buttons, light switches and the like. Viral pathogens can survive intact and in a condition capable of transmitting infection for substantial periods of time.

Medical literatures suggest that rhinoviruses, influenza viruses and adenoviruses are among the most important groups of pathologic agents that cause respiratory illness. Rhinoviruses, in particular, are thought to be the principal causative agents of upper respiratory infections, which are generally known as the common cold.

The rhinoviruses which generally causes the symptoms associated with the common cold are members of the picornavirus family. The viruses in this family lack an outer envelope and therefore are often characterized as “naked viruses”. Although more than 100 different antigenic types of rhinoviruses are known, all rhino viruses share certain central attributes. For example, all rhinoviruses are endowed with ether-resistant capsids. Rhinoviruses are acid mutable and contain single stranded RNA having a molecular weight of about 2.6·10⁶ Daltons. Rhinoviruses generally are difficult to inactivate or kill by the use of common antimicrobial compounds such as quaternary ammonium compounds or silver based antimicrobials.

Adenoviruses encompass more than thirty antigenic types. When adenoviruses invade the respiratory track, they cause inflammation of the respiratory tissues leading to symptoms of pharyngitis and bronchitis for example. Adenoviruses affect children much more commonly than adults. However, adenoviral infections of adults are not uncommon. In contrast to rhinoviruses, adenoviruses are DNA viruses and are not acid mutable. Rhinoviruses are similar to adenoviruses in that they lack an external envelope. Adenoviruses are unusually resistant to inactivation. G. Poli et al., Archiv Fur Lebensmittelhygiene, 29, 94-96 (1978). Poli et al. report a strain of adenoviruses to be susceptible to certain types of surface active agents (surfactants) which act as a disinfectant.

Parainfluenza viruses are members of the paramyxovirus family. Parainfluenza viruses play an important role in the occurrence of lower respiratory disease in children and upper respiratory disease in adults. These viruses include an ether sensitive lipoprotein envelope surrounding the nucleocapsid. Parainfluenza viruses are easier to inactivate than rhinoviruses or adenoviruses. They are rendered inactive by carboxylic acids even in low concentrations.

Research has demonstrated that citric, malic, pyretic and succinic acids among others are effective to inactivate herpes virus, orthomyxovirus and rhabdovirus. Poli, Biondi, Uberto, Balsari and Cantoni, [Poli et al.] Virucidal Activity of Organic Acids, Food Chem. (England)4(4)251-8 (1979). These experiments were performed at room temperature with aqueous solutions of pure acids. No substrate or carrier was used. The three viruses chosen for study by these workers were all viruses having envelopes similar to the parainfluenza 3 virus. Poli et al. also observed that the experimental group of acids was not effective in neutralizing adenovirus which is a naked virus. The researchers concluded that these acids were effective against enveloped viruses but not against naked viruses.

Thus, considerable benefit would be found in having a structure that could substitute for tissues or a handkerchief for small children and other individuals who are unable to free their hands for use in wiping or blowing their nose when they are suffering from an upper respiratory infection.

### SUMMARY OF THE INVENTION

The present invention solves many of the above discussed problems. The present invention generally includes an absorbent antimicrobial structure that can secured to the shoulder, arm, hand or wrist to provide a convenient surface to capture the products of coughing or sneezing or which can be used to wipe or blow the nose as necessary. In one example embodiment, the absorbent antimicrobial pad of the present invention, generally includes a multi layered absorbent structure including a cover, a virucidal agent, an absorbent core, an antimicrobial agent and an impermeable membrane secured to an attachment system by which the absorbent structure can be secured to the hand, arm or shoulder.

People suffering from a cold often suffer from an irritated nose as well, so that a dry absorbent structure placed against the skin on the nose may feel very uncomfortable. The present invention may include a lubricating agent applied to the surface of the absorbent structure to ameliorate this discomfort. For example, lubricants may include polyethylene glycol, polypropylene glycol or glycerin. This list of lubricants should not be considered to be limiting.

The present invention may also incorporate menthol or a similar substance to improve the comfort of the user. Menthol is non-prescription product which provides short term relief of minor sore throat and minor mouth or throat irritation. Menthol is commonly used in lip balms and cough medications. Further, menthol may reduce itching. An encapsulated form of menthol may be incorporated into the absorbent structure of the present invention.

In another aspect of the present invention, an antimicrobial agent may be incorporated into the absorbent structure to inhibit the growth of bacterial pathogens in or on the product, especially when the product may be used for an extended time. For example, acceptable antimicrobial compounds may include dimethyldiallyl ammonium chloride, trialkyl (p-vinylbenzyl) ammonium chloride and silver nitrate.

In another aspect of the invention, the absorbent structure may be formed in layers as follows: An absorbent and permeable covering layer incorporating a virucidal layer or component, a layer of encapsulated menthol, an absorbent core, a layer of an antimicrobial agent, an impermeable layer and finally an attachment system secured to the impermeable layer.

In one example of the present invention, the cover may be formed of a spun-laced material including polyester and cellulosic fibers. The basis weight of the fabric range, for example, between 60 and 80 grams per square meter. Appropriate materials may be available from commercial distributors PGI and BBA.

In another aspect of the present invention, increased softness of the cover material may be obtained by utilizing point bonded or resin bonded fiber blends.

In one aspect of the invention, the cover may be impregnated with virucide. Virucidal solution, optionally containing a softening agent and or a surfactant, may be applied to the cover fabric by spraying or printing on the cover. An exemplary solution composition includes: up to about 30% citric acid, about 10% polyethylene glycol 600, about 5% sodium lauryl sulfate and water.

In another aspect of the present invention, benzoic, succinic or malic acids may also be used as components of an effective virucidal material.

For example, about 12 milligrams per square inch of virucidal solution can be applied to the fabric by a conventional spraying technique.

Encapsulated menthol may be included beneath the cover. Encapsulated menthol capsules are pressure sensitive and can be broken by applying pressure to release the active ingredient, menthol. Other agents for comfort of the user may be incorporated as well.

In one aspect of the invention, encapsulated menthol can be sprayed onto the absorbent layer or onto the cover material. For example, encapsulated menthol may be applied at a rate of about 2 milligrams per square inch.

In one embodiment of the invention, absorbent materials for the absorbent core can be selected to include bonded cellulosic air laid material. For example, the basis weight of the air laid material may be between about 80 and 120 grams per square meter. Appropriate materials may be found commercially available from Buckeye Cellulose or Concert Industries.

The impermeable membrane may be formed, for example, from 1 mil thick polyethylene film. Other impermeable materials such as Mylar or metal foil may also be used. The absorbent core and cover materials may be glued or otherwise bonded to the impermeable membrane.

In another aspect of the invention, the attachment system may include pressure sensitive adhesives or double faced adhesive acetate tape applied to the impermeable membrane. The attachment system may also include a flap attachment, optionally including a resilient stretchable portion, which may be secured by adhesive or other means such as Velcro.

### BRIEF DESCRIPTION OF THE DRAWINGS

FIG. 1 is a perspective view of absorbent device in accordance with the present invention depicting two locations at which the absorbent device can be worn as worn by an individual;

FIG. 2 is a cross sectional view of an absorbent device in accordance with one embodiment of the invention;

FIG. 3 is cross sectional view of an absorbent device in accordance with another embodiment of the invention;

FIG. 4 is a plan view of an absorbent device in accordance with an embodiment of the invention; and

FIG. 5 is a plan view of another embodiment in accordance with the invention.

### DETAILED DESCRIPTION

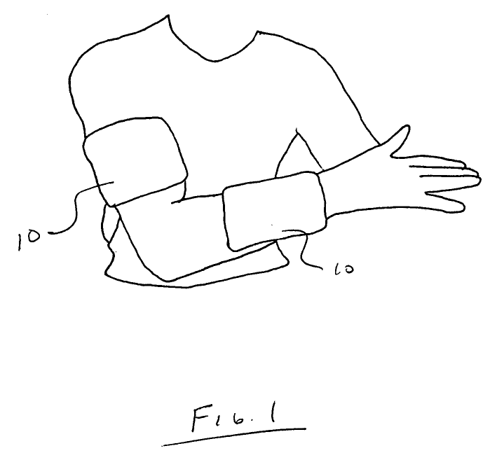

Referring to FIGS. 1-5, absorbent device 10 of the present invention generally includes cover 12, virucidal agent 14, absorbent core 16, antimicrobial agent 18, impermeable membrane 20 and attachment member 22 in an example embodiment of the invention.

Cover 12 may be formed of an absorbent porous soft material. For example, cover 12 may be formed of a spun laced material, including polyethylene and cellulosic fibers. The material for cover 12 may have a basis weight ranging between 60 and 80 grams per square meter. Appropriate materials are commercially available from PGI and BBA. If desired, increased softness of cover 12 may be obtained by utilizing a point bonded or resin bonded fiber blend.

In one aspect of the invention, virucidal agent 14 may be applied as a liquid. Virucidal agent 14 may optionally include a softening agent or a surfactant when it is applied to the fabric of cover 12. Virucidal agent 14 may be applied by spraying or printing onto cover 12 as well as by other techniques. While virucidal agent 14 is represented in the drawings as a separate layer one of skill in the art will recognize that virucidal agent 14 may be absorbed or impregnated into or onto another structural portion of the invention. Virucidal agent 14 may be a liquid, solid or gas and may also be chemically bonded to another component of the invention. Virucidal agent 14 may also be an inherent quality of a material chosen to form a structure of the invention.

In an example aspect of the invention, virucidal agent 14 may be an aqueous solution containing up to about 30% citric acid, about 10% polyethylene glycol 600, and 5% sodium lauryl sulfate.

In another embodiment of the invention, benzoic, carboxylic, succinic or malic acids may be utilized as components of virucidal agent 14. For example, virucidal agent 14 may be applied at a rate of about 12 milligrams per square inch to cover 12. Application may be by spraying, printing, dipping or other techniques known to those of skill in the art.

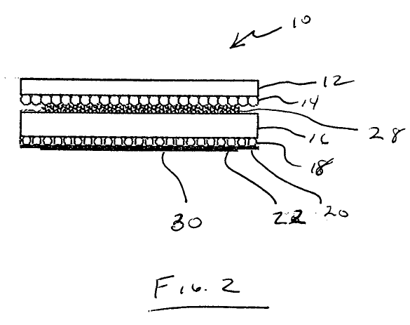

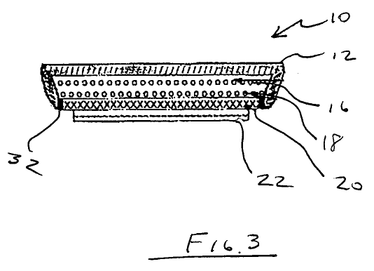

Referring to FIGS. 2 and 3, absorbent core 16 generally includes an absorbent material capable of absorbing nasal secretions and saliva and other materials that may be produced in a cough or sneeze. A variety of absorbent materials that can be used will be apparent to those of skill in the art.

For example, an example absorbent material is a bonded cellulosic air laid material having a basis weight of about 80 and 120 grams per square meter. Appropriate materials for absorbent core 16 are commercially available from Buckeye Cellulose and Concert Industries.

Antimicrobial agent 18 is adapted to prevent the growth of bacterial pathogens on or in absorbent device 10. Several classes of antimicrobial agents are readily available. Example antimicrobial agents 18 include antibiotics, disinfectants, alcohols and phenol. While antimicrobial agent 18 is represented in the drawings as a separate layer one of skill in the art will recognize that antimicrobial agent 18 may be absorbed or impregnated into or onto another structural portion of the invention. Antimicrobial agent 18 may be a liquid, solid or gas and may also be chemically bonded to another component of the invention. Antimicrobial agent 18 may also be an inherent quality of a material chosen to form a structure of the invention.

Impermeable membrane 20 may be formed of any liquid impermeable lightweight flexible material. For example, 1 mil thick polyether film is an appropriate material. Other materials that may appropriate include Mylar, metal foil and other polymeric vapor barrier materials.

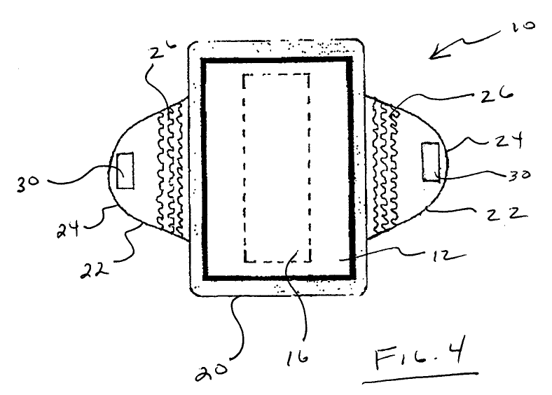

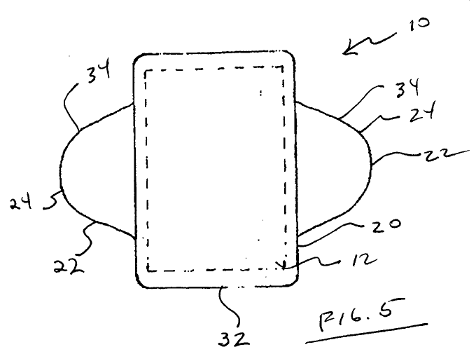

Referring to particularly to FIGS. 4 and 5, attachment member 22 may include a pressure sensitive adhesive or double faced adhesive tape such as acetate tape, which is applied to impermeable membrane 20. In addition, attachment member may include wings 24. Wings 24 may include a pressure sensitive adhesive appropriate for adhering to skin or fabric.

Wings 24 may also include elastic members 26. Wings 24 may be sized to wrap partially around an arm or other location to which absorbent 10 device may be secured or may be sized to wrap completely around the arm and be secured by Velcro or adhesive attachment of one wing 24 to another.

Referring to FIG. 2, an embodiment of absorbent device 10 is depicted in cross section. The depicted embodiment absorbent device 10 has a layered construction including cover 12, virucidal agent 14, absorbent core 16, antimicrobial agent 18, impermeable membrane 20 and attachment member 22.

Also depicted is menthol layer 28. Menthol layer 28 may be provided optionally to assist in short term relief of minor sore throat, mouth and throat irritation. Menthol also reduces itching which may be a particular benefit when the user is allergic. Menthol layer 28 may include encapsulated menthol which is encapsulated such that under pressure the capsules break to release the menthol within.

Referring to FIG. 3, another embodiment of absorbent device 10 is depicted. In this embodiment, absorbent device 10 includes cover 12, virucidal agent 14, absorbent core 16, antimicrobial agent 18, impermeable membrane 20 and attachment member 22. In this embodiment, attachment member 22 is formed from adhesive strips 30. Cover 12 and impermeable membrane 20 are joined along seam 32.

Referring to FIG. 4, in one embodiment of the invention, cover 12 and absorbent core 16 (shown in phantom) are depicted.

Referring to FIG. 5, a bottom plan view of an embodiment of absorbent device 10 is depicted. This embodiment includes alternate wing attachment 34.

In operation, absorbent device 10 is applied to a desired location such as a hand, wrist, forearm, upper arm or shoulder. When the user has the need to cough or sneeze, the user directs the cough or sneeze into absorbent device 10. When the user is a child, absorbent device 10 is placed on the forearm in the area where small children tend to habitually wipe their noses.

Absorbent device 10 should be replaced periodically to maintain the effectiveness of virucidal agent 14 and/or antimicrobial agent 18.

1. An absorbent device, comprising:
an absorbent portion;
an impermeable membrane operably coupled to the absorbent portion;
an attachment member operably coupled to the absorbent portion, the attachment member being adapted to couple the absorbent device to a human appendage; and
a virucidal agent effective to inactivate viruses in operable interaction with the absorbent portion such that/viruses that are deposited on or in the absorbent portion are deactivated by the virucidal agent.

2. The absorbent device as claimed in claim 1, further comprising an antimicrobial agent effective to kill bacterial pathogens.

3. The absorbent device as claimed in claim 1, wherein the absorbent portion comprises a cover and an absorbent core and wherein the virucidal agent is associated primarily with the cover.

4. The absorbent device as claimed in claim 1, further comprising menthol.

5. The absorbent device as claimed in claim 4, wherein the menthol is encapsulated in capsules such that the capsules are breakable under pressure to release the menthol.

6. The absorbent device as claimed in claim 1, wherein the attachment member comprises an adhesive effective to releasably attach the absorbent device to a human appendage or clothing covering a human appendage.

7. The absorbent device as claimed in claim 1, wherein the attachment member comprises wings to at least partially encircle the human appendage.

8. The absorbent device as claimed in claim 1, further comprising a substantially impermeable membrane coupled to the absorbent portion on one side thereof.

9. A method of capturing nasal or oral secretions that may include infective viruses, comprising:
securing an absorbent device to a human appendage in a position where the secretions can be directed into the absorbent device or a nose can be wiped on the absorbent device, the absorbent device comprising a virucidal agent effective to inactivate viruses that are deposited on or in the absorbent device; and
directing the nasal or oral secretions onto or into the absorbent device where the virucidal agent inactivates the virus.

10. The method as claimed in claim 9, further comprising applying an antimicrobial agent effective to kill bacterial pathogens to the absorbent device.

11. The method as claimed in claim 9, further comprising structuring the absorbent portion to comprise a cover and an absorbent core.

12. The method as claimed in claim 9, further comprising adding menthol to the absorbent device.

13. The method as claimed in claim 12, further comprising encapsulating the menthol such that the encapsulating capsules are breakable under pressure to release the menthol.

14. The method as claimed in claim 9, further comprising securing the absorbent device with an attachment member that includes an adhesive effective to releasably attach the absorbent device to a human appendage or clothing covering a human appendage.

15. The method as claimed in claim 9, further comprising equipping the attachment member with wings to at least partially encircle a human appendage.

16. The method as claimed in claim 9, further comprising attaching a substantially impermeable membrane to the absorbent portion on one side thereof.

17. An absorbent device, comprising:
means for absorbing nasal or oral secretions;
means for blocking passage of the nasal or oral secretions beyond the means for absorbing operably coupled to the means for absorbing;
means for attaching the absorbent device to a human appendage operably coupled to the means for absorbing and
means for inactivating viruses in operable interaction with the means for absorbing whereby viruses that are deposited on or in the absorbent device are deactivated.

18. The absorbent device as claimed in claim 17, further comprising means for killing bacterial pathogens.

19. The absorbent device as claimed in claim 17, wherein the means for attaching comprises an adhesive effective to releasably attach the absorbent device to a human appendage or clothing covering a human appendage.

20. The absorbent device as claimed in claim 17, further comprising means for at least partially encircling a human appendage.
